(19) United States
(12) Patent Application Publication
(10) Pub. No.: US 2002/0156059 A1
(43) Pub. Date: Oct. 24, 2002

(54) Pharmaceutical combination preparations that contain aromatase inhibitors and substances with estrogenic action as well as their use

(76) Inventor: Joerg Elliesen, Berlin (DE)

Correspondence Address: MILLEN, WHITE, ZELANO & BRANIGAN, P.C., 2200 CLARENDON BLVD., SUITE 1400, ARLINGTON, VA, 22201, US

(21) Appl. No.: 10/100,182

(22) Filed: Mar. 19, 2002

(30) Foreign Application Priority Data
Mar. 21, 2001 (DE) 10114522.5
Jul. 13, 2001 (DE) 10134768.5

(51) Int. Cl. A61K 31/56

(52) U.S. Cl. 514/182; 514/170

(57) ABSTRACT

The invention relates to pharmaceutical combination preparations that comprise aromatase inhibitors and substances with estrogenic action as well as their use for a selective estrogen replacement therapy (SERT), especially in the case of menopausal symptoms and the manifestations accompanying them. As substances with estrogenic action, estrogens, optionally also in combination with gestagens, are preferably used. The combination preparations according to the invention are preferably used in the case of menopausal symptoms, especially for treatment and prevention of postmenopausal osteoporosis and breast cancer as well as in other long-term sequelae that can be caused by estrogen deficiency.

The invention relates to pharmaceutical combination preparations that comprise aromatase inhibitors and substances with estrogenic action as well as their use for a selective estrogen replacement therapy (SERT), especially in the case of menopausal symptoms and the manifestations accompanying them. As substances with estrogenic action, estrogens, optionally also in combination with gestagens, are preferably used. The combination preparations according to the invention are preferably used in the case of menopausal symptoms and for prevention of postmenopausal osteoporosis, and in this case in contrast to long-term therapy with ERT/HRT alone, said preparations do not increase the risk of breast cancer since they prevent a stimulation of the mammary glands because of increased estrogen tissue concentrations.

The hormone replacement therapy for mitigating menopausal symptoms is a generally accepted treatment process, and in the case of short-term treatment, the ratio between treatment risk and success is clearly on the side of successful therapy.

For long-term therapies, an unambiguous assessment is not possible, however. Correlations between estrogen ingestion and cancer have always been discussed. In particular, the long-term treatment is associated with an increasing risk of breast cancer. This connection was observed in epidemiological studies with women who were treated over a period of 5 years and longer.

Estrogens, which are used many times against menopausal symptoms and after menopause for treatment of osteoporosis, thus stimulate the growth of breast tumor cells both in the premenopausal phase and in the postmenopausal phase.

The object of the invention was therefore to provide pharmaceutical preparations that allow a long-term treatment in the form of an estrogen replacement therapy and in this case do not increase or even reduce the risk of breast cancer.

The object of the invention is achieved by a combination preparation that consists of at least one aromatase inhibitor and at least one substance with estrogenic action. It has been shown that such a combination preparation can be used in a selective estrogen replacement therapy (SERT).

Even after menopause, the levels of estradiol in the breast tumor tissue are similar to those of tumors that occur before the ovarian function ceases, but the plasma-estrogen levels in postmenopausal females are 10 to 50 times lower than in premenopausal females (Yue, W. et al., Determinants of Tissue Estradiol Levels and Biologic Responsiveness in Breast Tumors. Breast Cancer Res Treat (Netherlands), 1998, 49 Suppl 1 pages 1-7, discussion pages 33-7).

The invention is based on findings that the estrogen content of the breast tissue is not determined just by the estrogen-plasma levels and a passive diffusion of the hormone, but rather is a result of active mechanisms such as an autonomous intracellular estrogen synthesis that consists of steroid hormone-precursor compounds (metabolic precursors). Estrogens in the postmenopausal female thus primarily originate from the aromatization of androgen-precursor compounds in peripheral fatty tissue. The specific hormonal situation of postmenopausal females with increasing production of ovarian and adrenal androgen-precursor compounds probably promotes the accumulation of newly synthesized estrogen in the breast tissue.

With the combination preparation according to the invention, on the one hand, the endogenic estrogen synthesis from androgen-precursor compounds in tissues with high aromatase activity, such as, e.g., breast tissue, is reduced by the use of at least one aromatase inhibitor.

In contrast, the accompanying hormone therapy with at least one substance that has an estrogenic action, on the one hand, prevents an additional estrogen deficiency, which would occur as a result of aromatase inhibition, and, on the other hand, keeps the estrogen-plasma level in menopause, especially in the case of postmenopausal women, high enough that the accompanying manifestations caused by the estrogen deficiency are suppressed.

In terms of this invention, aromatase inhibitors are all those compounds that prevent the formation of estrogens from their metabolic precursors by inhibition of the enzyme aromatase (inhibiting of biosynthesis). As aromatase inhibitors, therefore, all compounds are suitable that are suitable as substrates for the aromatase, such as, for example, the testolactone (17α-oxa-D-homoandrost-1,4-diene-3,17-dione) that is described in Journal of Clinical Endocrinology and Metabolism. 49, 672 (19979); the compounds androsta-4,6-diene-3,17-dione, androsta-4,6-dien-17β-ol-3-one-acetate, androsta-1,4,6-triene-3,17-dione, 4-androstene-19-chloro-3,17-dione, 4-androstene-3,6,17-trione that are described in “Endocrinology” 1973, Vol. 92, No. 3, page 874; the 19-alkynylated steroids that are described in DE-A1 31 24 780; the 10-(1,2-propadienyl)-steroids that are described in DE-A1 31 24 719; the 19-thioandrostrane derivatives that are described in EP-A1 0 100 566; the 4-androsten-4-ol-3,17-dione that is described in “Endocrinology” 1977, Vol. 100, No. 6, page 1684 and in U.S. Pat. No. 4,235,893, and esters thereof; the 1-methyl-15α-alkyl-androstra-1,4-diene-3,17-diones that are described in DE-A1 35 39 244, the 10β-alkinyl-4,9(11)-estradiene derivatives that are described in DE-A1 36 44 358, and the 1,2β-methylene-6-methylene-4-androstene-3,17-dione that is described in EP-A1 0 250 262.

Selective aromatase inhibitors are preferably used in the combination preparation according to the invention. Selective aromatase inhibitors are defined as compounds that act as substrate for the aromatase and in the dosage used, except for aromatase, do not influence any other enzyme in a clinically relevant way.

Considered as typical selective aromatase inhibitors are, for example, the steroidal compounds 1-methyl-androstra-1,4-diene-3,17-dione (DE-A1 33 22 285; atamestane), 4-hydroxy-4-androstene-3,17-dione (formestane) as well as the non-steroidal compounds (RS)-5-(4-cyanophenyl)-5,6,7,8-tetrahydro-imidazo-(1,5α)-pyridine, hydrochloride (Cancer Res., 48, pp. 834-838, 1988; fadrozole), 4-[cyano-α (1,2,4-triazol-1-yl)-benzyl]benzonitrile (CGS 20267), 5-[cyclopentylidene-(1-imidazolyl)-methyl]-thiophene-2-carbonitrile (EP-A1 0 411 735; pentrozole), 2,2′-[5-(1H′, 2′, 4-triazol-1-yl-methyl)-1,3-phenylene]-bis(2′-methylpropionitrile) (arimidex) as well as (6-[1-(4-chlorophenyl)-1,2,4-triazol-1-yl)-methyl]-1-methyl-1H-benzotriazole, dihydrochloride (vorozole).

The list of selective aromatase inhibitors is not complete; also other compounds that are mentioned in the cited publications as well as other compounds that correspond to the requirements are suitable.

They are produced and manufactured according to processes that are known in the art and are available, depending on the desired application, preferably in oral form, as an injection or as a long-term preparation. The aromatase inhibitors according to the invention can be administered as single doses or as depot forms.

An excessive load of the mammary gland tissue by estrogens from the metabolic conversion of androgens is avoided by the specific administration of a preferably selective aromatase inhibitor. This is especially important in postmenopause, since androgens are increasingly formed within the framework of counter-regulation because of the ovarian estrogen deficiency. The simultaneous decrease in SHBG, a binding protein for steroid hormones, increases the proportion of free androgens in the plasma and promotes an increase in androgen concentration in the mammary gland tissue. A hormone substitution treatment can also produce a reduction in the SHBG level, especially if the estrogen is combined with a gestagen that has partial androgenic actions.

In general, the daily dosages for an aromatase inhibitor are 100 mg-600 mg. With a daily dose of 200 mg, the target area is generally well covered.

As substances with estrogenic action in terms of the invention, all known substances can be used that can be used for standard hormone replacement therapy in menopause to correct symptoms of estrogen deficit. These are preferably preparations that have estrogens or preparations that have estrogens and gestagens.

As estrogens, all natural and synthetic compounds that are known as estrogenically active are suitable.

As natural estrogens, these are in particular estradiol as well as its esters that have a longer action, such as valerate, etc., or estriol. In addition, synthetic estrogens such as ethinylestradiol, 14α,17α-ethano-1,3,5(10)-estratriene-3,17β-diol, 14α,17α-ethano-1,3,5(10)estratriene-3, 16α,17β-triol or the 15,15-dialkyl derivatives of estradiol can be mentioned.

Estratriene-3-amidosulfonates, derived from estradiol or ethinylestradiol, as well as 14α,17α-methylene steroids from the estrane series and the corresponding 3-amidosulfonate derivatives can also be mentioned.

Preparations that contain estrogens in combination with gestagens or have gestagens alone are also extensively known.

Gestagens are preferably selected from the group of compounds:

Gestodene, progesterone, desogestrel, 3-ketodesogestrel, levonorgestrel, lynestrenol, norgestimate, norethisterone, norethisterone acetate, chlormadinone acetate, drospironenone, cyproterone acetate or dienogest.

Estrogens and gestagens are also present, of course, as combination preparations, thus, e.g., as single-phase preparations or as graduated combination preparations or else as sequence preparations (estrogen (first phase) and estrogen/gestagen (second phase) and are suitable according to the invention.

The list of usable estrogens and gestagens is not complete; other compounds that meet the requirements are also considered.

The substances with estrogenic action are administered in standard dosages (e.g., estradiol orally 0.5-2.0 mg/day, conjugated estrogens 0.3-1.25 mg/day) to offset the estradiol-plasma levels and to maintain a premenopausal level. In the individual case, higher dosages are also administered.

Suitable dosages are set depending on body weight, age and constitution of the patient, whereby the necessary daily dose can be administered one or more times.

The substances with estrogenic action can be administered intravenously, subcutaneously, intramuscularly, orally, intranasally or intravaginally.

The administration to the patients of aromatase inhibitors and substances that have an estrogenic action can be carried out simultaneously and/or sequentially in time. It takes into consideration the kinetics of the individual substances and is selected such that active plasma levels of the aromatase inhibitor, like the hormones used for hormone replacement therapy, are achieved. Simultaneously and sequential in time, this means that the aromatase inhibitor is optionally administered over a certain period and then the treatment is continued with aromatase inhibitors and a substance or substances that has or have an estrogenic action.

The pharmaceutical combination preparation according to the invention is suitable for a selective estrogen replacement therapy, especially for a long-term treatment in the case of menopausal symptoms, preferably in the postmenopausal phase. On the one hand, accompanying manifestations of the menopause that occur, such as, e.g., osteoporosis, are avoided, and possible lost bone mass is again built up, and, on the other hand, the risk of breast cancer is simultaneously reduced.

The subject of the invention is therefore also the use of the combination preparation according to the invention for a selective estrogen replacement therapy (SERT), especially for treating menopausal symptoms, preferably for long-term therapy of postmenopausal accompanying manifestations that can be attributed to estrogen deficiency.

The pharmaceutical combination preparation according to the invention is produced by the aromatase inhibitors and substances with estrogenic action being formulated together or separately from one another with commonly used pharmaceutical vehicles, adjuvants and/or additives, whereby the dispensing forms of the individual active ingredients must not be identical. It is eminently possible, e.g., that one active ingredient of the combination preparation is administered orally, while the other active ingredient is administered subcutaneously.

These pharmaceutical compositions and combination preparations can be provided for oral, rectal, vaginal, subcutaneous, percutaneous, intravenous or intramuscular administration.

The combination preparations of the invention are produced with the commonly used solid or liquid vehicles or diluents and the commonly used pharmaceutical-technical adjuvants corresponding to the desired type of administration with a suitable dosage in a known way. The preferred preparations consist in a dispensing form that is suitable for oral administration. Such dispensing forms are, for example, tablets, film tablets, coated tablets, capsules, pills, powder, solutions or suspensions or else depot forms.

Of course, parenteral preparations such as injection solutions can also be considered. In addition, for example, suppositories and agents for vaginal use can also be mentioned as preparations.

Corresponding tablets can be obtained by, for example, mixing the active ingredient with known adjuvants, for example inert diluents such as dextrose, sugar, sorbitol, mannitol, polyvinyl pyrrolidone, explosives such as corn starch or alginic acid, binders such as starch or gelatin, lubricating agents such as magnesium stearate or talc and/or agents for achieving a depot effect such as carboxylpolymethylene, carboxylmethyl cellulose, cellulose acetate phthalate or polyvinyl acetate. The tablets can also consist of several layers.

Correspondingly, coated tablets can be produced by coating nuclei that are produced analogously to the tablets with agents that are commonly used in tablet coatings, for example polyvinyl pyrrolidone or shellac, gum arabic, talc, titanium oxide or sugar. In this case, the coated tablet shell can also consist of several layers, whereby the adjuvants that are mentioned above in the tablets can be used.

The solutions or suspensions can contain additional taste-improving agents such as saccharin, cyclamate or sugar, as well as, e.g., flavoring substances such as vanilla or orange extract. In addition, they can contain suspending adjuvants such as sodium carboxymethyl cellulose or preservatives such as p-hydroxybenzoates.

In addition, capsules can be produced, for example, by inert vehicles such as lactose or sorbitol being added and encapsulated in gelatin capsules.

Suitable suppositories can be produced by, for example, mixing with vehicles that are provided for this purpose, such as neutral fats or polyethylene glycol or derivatives thereof.

The subject of the invention is also the packaging unit, which comprises at least two components. It contains the active ingredients that are manufactured together or separated in space and as a further component directions on simultaneous administration and/or administration that is sequential in time of the dispensing forms.

Below, the invention is to be explained in more detail by examples without being limited thereto, however.

### EXAMPLE 1

Combination preparation for women in menopause who do not require endometrium protection by gestagens (e.g., after a hysterectomy) for continuous daily administration:

a) Estrogen Preparation Daily dosage unit based on the preparation, e.g., 1-2 mg of estradiol-17β or 6.25 mg of a conjugated estrogen in one dose and

b) Aromatase Inhibitors e.g., 200 mg of atamestane in one dose.

### EXAMPLE 2

Combination preparation for perimenopausal women with intact uteri for a 28-day cycle:

a) Estrogen Preparation Daily dosage unit based on the preparation, e.g., 1-2 mg of estradiol-17β or 0.625 mg of a conjugated estrogen in one dose,

b) Gestagen preparation for 10-14 days (sequential addition) Daily dosage unit based on the respective gestagen preparation, e.g., 5 mg of MPA, 1 mg of NETA, 75-150 μg of LNG, 1 mg of CPA or 50 μg of gestodene in one dose, and

c) Aromatase Inhibitor e.g., 200 mg of atamestane in one dose.

### EXAMPLE 3

Combination preparation for postmenopausal women who have not undergone a hysterectomy for continuous daily administration:

a) Estrogen Preparation Daily dosage unit based on the preparation, e.g., 1-2 mg of estradiol-17β or 0.625 mg of a conjugated estrogen in one dose,

b) Gestagen Preparation Daily dosage unit based on the respective gestagen preparation, e.g., 5 mg of MPA, 1 mg of NETA, 75-150 μg of LNG, 1 mg of CPA or 50 μg of gestodene or less in one dose, and

c) Aromatase Inhibitor e.g., 200 mg of atamestane in one dose.

List of Abbreviations

MPA=medroxyprogesterone acetate

NETA=norethisterone acetate

LNG=levonorgestrel

CPA=cyproterone acetate

1. Pharmaceutical combination preparation that comprises at least one aromatase inhibitor and at least one substance with estrogenic action.

2. Combination preparation according to claim 1, characterized in that the aromatase inhibitor is a selective aromatase inhibitor, preferably atamestane, formestane, pentrozole, arimidex, fadrozole, CGS 20267 or vorozole.

3. Combination preparation according to claim 1 or 2, wherein the substance with estrogenic action represents an estrogen or an estrogen in combination with a gestagen.

4. Combination preparation according to claim 3, wherein the estrogen is selected from the group of natural estrogens or the synthetic estrogens or derivatives thereof.

5. Combination preparation according to claim 3, wherein the gestagen is selected from the group of compounds gestodene, progesterone, desogestrel, 3-ketodesogestrel, levonorgestrel, lynestrenol, norgestimate, norethisterone, norethisterone acetate, chlormadinone acetate, drospironenone, cyproterone acetate or dienogest.

6. Process for the production of a pharmaceutical combination preparation according to one of claims 1-5, wherein at least one aromatase inhibitor and a substance with estrogenic action are formulated together or separately from one another with commonly used pharmaceutical vehicles, adjuvants and/or additives.

7. Use of at least one aromatase inhibitor and at least one substance with estrogenic action for selective estrogen replacement therapy.

8. Use according to claim 7 for treatment of menopausal symptoms, especially for long-term therapy of postmenopausal accompanying manifestations, which can be attributed to estrogen deficiency.
